(19) United States
(12) Patent Application Publication
(10) Pub. No.: US 2003/0129616 A1
(43) Pub. Date: Jul. 10, 2003

(54) Use of a mutation in the gene for the beta3-subunit of human g-protein

(76) Inventor: Winfried Siffert, Essen (DE)

Correspondence Address: NIXON PEABODY, LLP, 8180 GREENSBORO DRIVE, SUITE 800, MCLEAN, VA, 22102, US

(21) Appl. No.: 10/203,009

(22) PCT Filed: Feb. 5, 2001
(86) PCT No.: PCT/EP01/01196

(30) Foreign Application Priority Data
Feb. 3, 2000 (DE) 100-04-681.9
Feb. 21, 2000 (DE) 100-07-587.8
Jun. 24, 2000 (DE) 100-30-945.3

(51) Int. Cl. C12Q 1/68; C07H 21/04

(52) U.S. Cl. 435/6; 536/23.2

(57) ABSTRACT

The invention relates to use of a gene change in the gene for the β33 subunit of the human G protein, in exon 9 at position 657 in appendix 1 there being a substitution of adenine by thymine and/or in exon 10 at position 814 in appendix 1 there being a substitution of guanine by adenine and/or in the promotor area at position (−350) in appendix 1 there being a substitution of adenine by guanine and/or in intron 9 at position 3882 in appendix 1 there being a substitution of adenine by cytosine and/or in intron 9 at position 5177 in appendix 1 there being a substitution of guanine by adenine and/or in intron 9 at position 5249 in appendix 1 there being a substitution of guanine by adenine and/or in intron 10 at position 6496 in appendix 1 there being an insert consisting of the nucleotides cytosine-adenine-cytosine-adenine, for predicting physiological and pathophysiological processes in the human body.

The invention relates to new sequence variants of the human gene GNB3 which codes for the Gβ3 subunit of human G proteins and their use for predicting physiological and pathophysiological processes in the human body, especially for diagnosis of a wide spectrum of diseases, especially for establishing a disposition to high blood pressure and overweight, a disposition to a host of conditions which accompany faulty control of the immune system, a disposition to psychiatric conditions, as well as development of therapeutic agents and dedicated use of known drugs and those still to be developed based on pharmacogenetic principles.

The human Gβ3 subunit is an important component in signal transmission systems which occur in all human cells. The sequence variations in GNB3 are associated with increased occurrence of the G protein β3 splice variants Gβ3s and Gβ3s2. They are responsible for increased signal transmission in cells which express Gβ3s and Gβ3s2.

Since Gβ3 occurs in all cells which have been studied for a long time, the increased signal transmission mediated by Gβ3s and Gβ3s2 leads to an increase of the physiological and pathophysiological responses to a host of nonhormonal and hormonal stimuli.

This function change makes itself noticeable in a wide spectrum of central-nervous and peripheral functions, thus in the cardiovascular system, in metabolic functions, central nervous functions, and neurosecretion. Thus, for example Gβ3, Gβ3s and Gβ3s2 are interesting points of approach for drugs and therapeutic agents with a wide indication focus.

Diverse findings point to Gβ3, Gβ3s and Gβ3s2 playing a part in the pathogenesis/pathophysiology of a series of common conditions, for example hypertonia, cardiac infarction, coronary heart disease and other cardiovascular conditions, stroke, and metabolic diseases such as obesity and diabetes. Furthermore they play a part in other dysfunctions, such as for example erectile dysfunction. The variations are especially involved in changes of immune regulation and are therefore suitable for predicting a disposition to or the course of a host of immunological conditions in which increased or reduced function of the immune cells is important. These conditions include among others HIV infection, asthma, psoriasis, so-called atopic conditions such as atopic dermatitis, hepatitis B and hepatitis C, transplant rejection, Crohn's disease, ulcerous colitis, and many others. In addition, these variations play a part in preeclampsia/gestosis, spontaneous abortion, the success of in vitro fertilization, etc.

The object of the invention is to determine variations, polymorphisms, mutations and the resulting haplotypes in the DNA sequence of the human Gβ3 gene (GNB3) and to ascertain their correlation with disease dispositions. Proceeding from this correlation, a process for diagnosis of these disease dispositions, for prediction of the severity, the course and survival time, a system for prediction of the individual responsiveness to various drug classes and a system for development of a new class of Gβ3 or Gβ3s and Gβ3s2 effective therapeutic agents, and the development of test systems for studying pathophysiological relationships and development of the aforementioned therapeutic agents will be developed. In summary, for each GNB3 genotype an individually optimum therapeutic agent can be predicted or developed.

Importance of Altered Activation of G Proteins for Different Diseases

Human heterotrimeric G proteins are composed of the subunits α, β, and γ. Of them, in turn a series of isoforms is known which are coded by different genes. For example there are 13 different γ isoforms (γ1- γ13), at least 5 different β isoforms (β1-β5) and a host of different α isoforms (αs (short and long), αo, αi1-3, αq, α11-16, αolf, etc).

Since G proteins are recognized to play a central part in the control of the function of all human cells, regardless of which cell receptors are activated, it can be directly expected that the course of manifold and quite different diseases is influenced in the genetically determined, intensified activatability of G proteins. In the diverse functions of G proteins, function-changing mutations acquire especially outstanding importance and prediction capacity. This is in contrast to mutations in other genes which code for other proteins, for example, hormones or hormone receptors.

Two examples will be used to illustrate this:

If a function-changing mutation is found in opioid receptors, one skilled in the art will first think of reduced or intensified action of exogenously supplied opiates or intensified or reduced opioid dependency. Furthermore, behavior, pain, etc. could be modulated. In no case would one skilled in the art think immediately that mutations in opioid receptors would affect the course of HIV infection or tumor progression in a lasting manner.

If a mutation occurs in the visual pigment rhodopsin or for example in the G protein—G_{olf} which is expressed only in the human olfactory organ, due to the extreme limitation of expression of the gene products in the eye or nose only an effect on seeing or smelling can be expected, but no relationship to cardiovascular diseases.

This means accordingly that in contrast to the indicated examples, by gene changes in proteins—such as G proteins—which are expressed in all cells of the human body, and which regulate cell functions there at a central location, all physiological and pathophysiological processes are decisively influenced or at least modulated.

In the scientific literature it has been repeatedly postulated that function changes of G proteins have a lasting effect on diverse diseases or the course of diverse diseases. These gene changes can be structure-changing mutations in the G protein subunits which for example change the activation capacity by the receptor or the dimerization of βγ subunits. In addition, these changes could change the composition of heterotrimeric G proteins. Furthermore, the expression level of these G protein subunits could be changed. Some examples will also be given here:

Importance of Altered G Protein Activation in Psychiatric Conditions such as Depression, Anxiety, and Schizophrenia

It has been known for years that in these conditions there is altered intracellular signal transduction which could be caused by an altered G protein function. Furthermore there is evidence that the success of for example antidepressive therapy leads to altered G protein functions. Diverse publications from the literature can be cited in this regard, for example:

Odagaki Y, Koyama T, Yamashjita I. Platelet pertussis toxin-sensitive G proteins in affective disorders. J Affect Disord. July 1994; 31 (3); 173-7.

Garcia-Sevilla J A, Walzer C, Busquets X, Escriba P V, Balant L, Guimon J. Density of guanine nucleotide-binding proteins in platelets of patients with major depression: increased abundance of the Gαi2 subunit and down-regulation by antidepressant drug treatment. Biol Psychiatry. Oct. 15, 1997; 42 (8): 704-12

Pacheco M A, Stockmeier C, Meltzer H Y, Overholser J C, Dilley G E, Jope R S. Alterations in phosphoinositide signalling and G-protein levels in depressed suicide brain. Brain Res. Jun. 3, 1996; 723(1-2): 37-45.

These selected examples prove that for many years the relationship between G protein activity and affective diseases has been known. In any case these studies focussed solely on studies of possible gene changes in the α subunits or on changes of expression of these G protein α subunits. A relationship between these diseases and altered function of β or γ subunits was not assumed.

Importance of Altered G Protein Activation in Inflammatory Conditions, Infectious Diseases, and Autoimmune Diseases (Arthritis, Asthma, Psoriasis, HIV, Hepatitis, etc.)

In these diseases, as is recognized, the activity and activation capacity of cells of the immune system (lymphocytes, granulocytes, etc.) play an outstanding part in the course of the disease. The activation of these cells is controlled in turn by pertussis toxin-sensitive G proteins.

Examples from the Scientific Literature are:

Thomazzi S M, Souza M H, Mel-Filho A A, Hewlett E L, Lima A A, Ribeiro R A. Pertussis toxin from Bordetella pertussis blocks neutrophil migration and neutrophil-dependent edema in response to inflammation: Braz J Med Biol Res. January 1995; 28 (1): 120-4

Stanley J B, Gorczynski R M, Delovitch T L, Mills G B. IL-2 secretion is pertussis toxin sensitive in a T lymphocyte hybridoma. J Immunol. May 15, 1989; 142(10): 3546-52.

Bacon K B, Camp R D. Interleukin (IL)-8-induced in vitro human lymphocyte migration is inhibited by cholera and pertussis toxins and inhibitors of protein kinase C. Biochem Biophys Res Commun. Jun. 29, 1990; 169(3): 1099-104.

I. H. Chowdhury, Y. Koyanagi, 0. Hazeki, M. Ui and N. Yamamoto. Pertussis toxin inhibits induction of human immunodeficiency virus type 1 in infected monocytes. Virology 203 (2): 378-383, 1994.

If therefore there is increased activation capacity of G proteins genetically induced, it can be expected that activation of leukocytes, their proliferation and migration, the release of inflammation mediators, etc. are likewise increased. Thus, for the affected individual there is fundamentally higher readiness to contracting diseases with altered immune function, or having a different disease course.

In this application it is novel in any case that in contrast to general expectations decisive gene changes in gene GNB3 are described, to which the indicated effects can be attributed and which codes for the β3 subunit of heterotrimeric G proteins.

Tumors/Cancer

Altered G proteins can on the one hand allow degeneration of malignant cells. On the other hand, any cancer disease is characterized by proliferation of tumor cells and metastasis, since proliferation and cell migration are G protein-mediated processes. Thus altered G protein activation must have affects on the course of tumor conditions.

Examples from the Scientific Literature are:

J. Lyons, C. A. Landis, G. Harsh, L. Vallar, K. Gruenewald, H. Feichtinger, Q. -Y. Duh, O. H. Clark, E. Kawasaki, H. Bourne and F. McCormick. Two G protein oncogenes in human endocrine tumors. Science 249: 655-659, 1990.

L. S. Weinstein and A. Shenker. G protein mutation in human disease. Clin. Biochem. 26: 333-338, 1993.

Vallar L. Oncogenic role of heterotrimeric G proteins. Cancer Surv. 1996; 27: 325-38. Review.

Pace A M, Wong Y H, Bourne H R. A mutant α subunit of Gi2 induces neoplastic transformation of Rat-1 cells. Proc Natl Acad Sci USA. Aug. 15, 1991; 88(16): 7031-5

For a long time in the scientific literature solely α-subunits of heterotrimeric G proteins had been studied for whether mutations contribute causally or by modulation to cancer conditions. In no known publication has it been postulated that these causally effective gene changes or gene changes which influence the course of the disease could also be present in β-subunits of heterotrimeric G proteins.

Furthermore, in the scientific literature there are references to the fact that the importance of G proteins has been postulated in general for the formation of the disease and the course of the disease.

Weinstein and Shenker wrote in a survey work in 1993 (S. Weinstein and A. Shenker. G. protein mutations in human disease. Clin. Biochem. 26:333-338, 1993.): “Heterotrimeric G proteins are coupled on cell surface receptors which mediate extracellular signals to intracellular effectors which produce secondary messenger substances. Disrupted G protein signal relay which is caused by posttranslational modification by bacteria toxins, altered gene expression or gene mutations, leads to diverse biological consequences. Mutations within a gene which codes for a G protein subunit which lead either to a constitutional activation or a loss of function have been identified. These G protein mutations play a part in the pathogenesis of various human diseases, including in some endocrine tumors, in McCune/Albright syndrome and hereditary osteodystrophy (Albright syndrome).”

This survey article thus relates in turn only to ascertained somatic mutations in α-subunits of heterotrimeric G proteins.

Spiegel A M wrote in 1997 (Inborn errors of signal transduction: mutations in G proteins and G protein-coupled receptors as a cause of disease. J Inherit Metab Dis June 1997; 20(2) 113-21): “A wide spectrum of neurotransmitters, polypeptide hormones and other molecules use G protein-coupled paths to transmembrane signal transduction. In recent years mutations in G protein-coupled receptors in α-subunits of G proteins as a cause of a host of human diseases have been identified. Mutations which led to loss or intensification of functions were described in diseases such as hereditary Albright's osteodystrophy, nephrogenic insipid diabetes, McCune-Albright syndrome and in male precocious puberty. Identification of mutations in G protein coupled receptors and in G proteins in human diseases provided unique insight into G protein-coupled signal transduction, important implications for diagnosis and possible treatments and should advance the search for additional defects in G protein-coupled signal transduction for other diseases.”

This work also described the outstanding role of G proteins for diverse diseases, but in turn emphasizes solely the importance of a α-subunits.

Lefkowitz wrote in 1995: (R. J. Lefkowitz. G proteins in medicine. N. Engl. J. Med. 332: 186-187, 1995):

“If the ubiquitous distribution of G proteins and the notable diversity of their functions are examined, it should not be surprising that changes in the structure or the expression of G proteins can lead to extremely serious pathophysiological consequences. These changes lead either to an increase or decrease of activity of the affected G proteins.”

Increased G Protein Activity and Risk for Bronchial Asthma

In the presence of an increased activation capacity of G proteins and concomitant increased activation capacity of immune cells, an increased risk of 825T allele carriers to contract asthma can be assumed.

In this disease of the respiratory passages there is often a hyperreactive bronchial system, and activation of immune cells with increased migration and increased secretion of antibodies for example of the type IgE is generally known. Last but not least, due to this importance of the immune system for the occurrence and severity of bronchial asthma, therapy optionally also includes the use of glucocorticoids for suppression of the immune response or leukotriene receptor antagonists for reducing leucocytene activation.

It was studied whether in children with asthma, compared to a healthy control collective, gene changes in the gene GNB3 increasingly occur. To do this, 101 children with bronchial asthma were genotyped with respect to the C825T polymorphism in gene GNB3 and compared to 332 healthy control individuals who had no bronchial asthma through age 25. In children with bronchial asthma, at the 825 locus the following genotype distribution was found: 13 TT (13%), 54 TC (53%) and 34 CC (34%). The 825T allele frequency is thus 39.4%. This high 825T allele frequency within a purely Caucasian cohort is already conspicuous per se and is similar to that which is found otherwise only in East Asians (Siffert et al., 1999; J. Am. Soc. Nephrol. Vol. 10, 1921-1930). In contrast, in the 332 healthy controls the 825T allele frequency with 29.7% was clearly reduced and the determined genotype distribution yielded 25 TT (7.5%), 147 TC (44.3%) and 160 CC (48.2%). The difference of the genotype distribution compared to patients with bronchial asthma is statistically significant (chi² test; p=0.023; chi2=7.6, 2 degrees of freedom). Thus the asthma risk for a homozygotic 825T allele carrier (TT genotype) compared to the CC genotype is increased by a factor of 2.4 (p=0.019) and for a heterozygotic 825T allele carrier (TC genotype) is increased by a factor of 1.7 (p=0.0257).

Increased G Protein Activation and Therapy Response to the Administration of Interferon Plus Ribavirin in Patients with Hepatitis C Infection

After infection with the hepatitis C virus (HCV) many patients can spontaneously eliminate the virus, while in other patients the virus infection persists. Typical late consequences in patients with persistent HCV infection are the formation of hepatic cirrhosis, possibly with hepatic cell carcinoma. In decompensation of the disease, fatal liver failure can possibly occur. Some of these patients receive liver transplants. In addition, the attempt is made to eliminate HCV by medication, for example by combined administration of ribavirin, which suppresses HCV replication, in combination with the administration of interferons. The success of this therapy is continuously monitored via the determination of the number of HCV copies in the serum. In roughly 50-70% of patients this therapy leads to continuing elimination of HCV and these patients are called “sustained-responders”. A series of patients does not respond to this therapy or does so only at its start, with a subsequent re-increase of the virus burden. These patients are called “non-responders” or patients with “relapse”.

It was studied whether gene changes in gene GNB3 can be used for predicting the success of combination therapy with ribavirin and interferon. To do this, 85 HCV patients were genotyped with respect to the C825T polymorphism. In the patients with “sustained response” the following genotype distribution was observed: 8 TT, 37 TC, 39 CC. In the “non-responders” the corresponding genotype distribution was 1 TT, 12 TC, 17 CC. Thus, among the “responders” compared to the “nonresponders” there is a significantly increased accumulation of the 825T allele (p=0.016). In homozygotic 825T allele carriers (TT genotype) there are 89% responders, in heterozygotic 825T allele carrier (TC genotype) 68% responders, in homozygotic C825 allele carriers (CC genotype) on the other hand there are only 31% responders. For patients with the TT genotype compared to the CC genotype this yields a probability which is increased by a factor of 11.3 of belonging to the group of responders (p=0.012) and for patients with the TC genotype compared to the CC genotype the probability of belonging to the “responders” is increased by a factor of 2.9 (p=0.03). This example illustrates that gene changes in gene GNB3 are especially suited for predicting the response to certain drugs or the success or failure of therapy.

The Examples Explained Above Yield the Following:

1. Gene changes in the genes which code for ubiquitously expressed proteins influence diverse diseases and cause diverse disease risks

2. G proteins control almost all signal transduction processes in the human body

3. Changes in G proteins have diverse effects in quite different diseases. They can promote the formation of diseases or influence their course

4. While the cited literature clearly indicates that in general it can be assumed that G protein mutations can cause these diseases, a relationship to β or γ subunits of heterotrimeric G proteins with disease risks has neither been described nor assumed in the literature.

Thus, the relationships described below between gene changes in gene GNB3 and various diseases or disease courses or the use of these gene changes for predicting diseases and disease courses and for predicting the reaction to drugs are on a scientifically reproducible foundation. What is new and unexpected is the fact that this predictability can be accomplished by gene changes in specifically this gene (GNB3).

Structure of the Gene GNB3

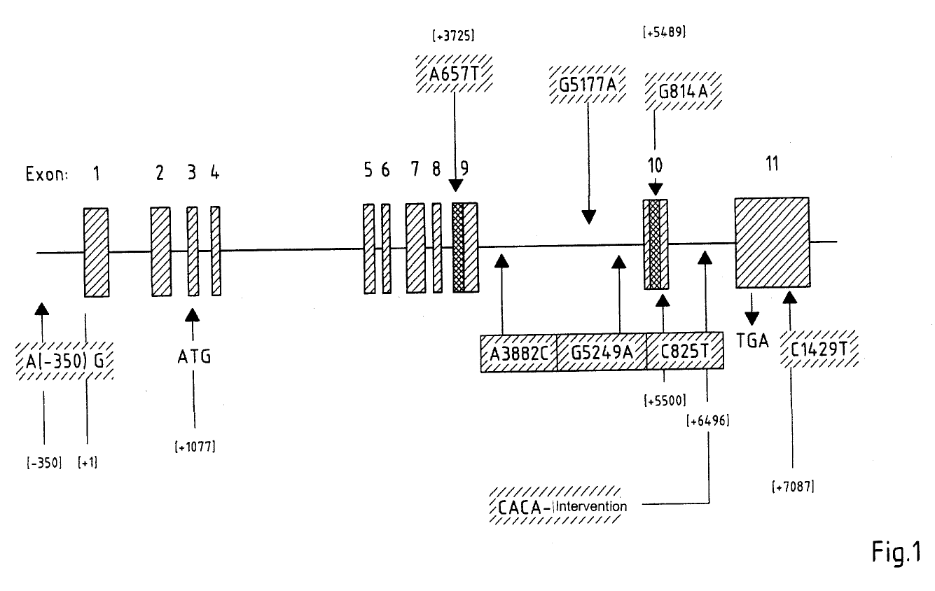

The structure of the gene GNB3 is shown in FIG. 1. It consists of 11 exons, the start codon ATG being located in exon 3, while the stop codon is located in exon 11. The cDNA which codes for the Gβ3 protein has already been described (M. A. Levine, P. M. Smallwood, P. T. Moen, Jr., L. J. Helman and T. G. Ahn. Molecular cloning of beta 3 subunit, a third form of the G protein beta-subunit polypeptide. Proc.Natl.Acad. Sci USA 87 (6):2329-2333, 1990). In contrast to this published cDNA sequence, the applicant previously described mutations C825T (FIG. 1) and C1492T (FIG. 1) (compare DE 196 19 362 A1, DE 196 37 518 A1 and PCT/EP99/06534 published later). The numbering of the aforementioned polymorphisms relates to the cDNA sequence, position +1 being assigned to the start codon ATG. Moreover, it has already been described that alternative splicing of exon 9 yields a new splice version with 6 WD repeats (Gβ3s) (W. Siffert, D. Rosskopf, G. Siffert, S. Busch, A. Moritz, R. Erbel, A. M. Sharma, E. Ritz, H. E. Wichmann, K. H. Jakobs, and B. Horsthemke. Association of a human G protein β3 subunit variant with hypertension. Nat. Genet. 18 (1):45-48, 1998) and that alternative splicing of exon 10 leads to formation of another splice variant with 6WD repeats (compare PCT/EP99/06534). It was found that the 825T allele and the 1492T allele are predictive for the generation of these splice variants. Since the expression of these splice variants in cells generally leads to increased activation capacity of G proteins, the strength of intracellular signal transduction can be predicted via genotyping of the C825T or C1429T polymorphism.

In addition, the genomic sequence of GNB3 has been published (M. A. Ansari-Lari, D. M. Muzny, J. Lu, F. Lu, C. E. Lilley, S. Spanos, T. Malley, and R. A. Gibbs. A gene-rich cluster between the CD4 and triosephosphate isomerase genes at human chromosome 12p13. Genome Research 6: 314-326, 1996)

It has been found that in intron 9 the sequence of the human Gβ3 gene (GNB3) in contrast to the sequence described by Ansari-Lari et al., in addition to the mutations in the cDNA (C825T, A657T, G814A, C1429T) and a mutation in the promotor (A-350G), there are other variants. It was otherwise found that these genetic variants correlate with a disposition to various diseases, among others, high blood pressure, obesity, overweight.

The subject matter of the invention is accordingly the sequence of the human Gβ3 gene which is completely or partially mutated at positions 657, 814, 825 and 1429 of the cDNA or at positions (−350), 3882, 5177, 5249 and 6496 of the genomic sequence.

FIG. 1 of the drawings shows the gene structure of GNB3 and new polymorphisms.

The exon-intron structure of GNB3 is described. The gene consists of 11 exons, previously described polymorphisms are exon 10 (C825T) and exon 11 (C1429T). The numbering corresponds to the cDNA sequence, position +1 being assigned to the start codon ATG in exon 3. Furthermore, the start of transcription in exon 1 is described. Relative to this transcription start point, a mutation can be found in the promotor (A-350G) and the described mutations can be found in intron 9, A3882C, G5177A, and G5249A. Using this numbering (transcription start point=1) the C825T replacement is in position 5500. Furthermore CACA insertion can be found at position 6496.

Furthermore, the areas of exon 9 and exon 10 which are alternatively spliced when mutations are present at positions 825 and 1429 of the cDNA and 3882, 5177, 5249 and/or 6496 of the DNA sequence are shown. The numbering of polymorphisms is shown in square brackets when the transcription start point in exon 1 is fixed as position 1.

In particular, the mutations are characterized below. The numbering relates to the transcription start point of GNB3 (FIG. 1). This start point corresponds to nucleotide 52221 of the sequence of a section of the human chromosome 12 published by Ansari-Lari et al. (M. A. Ansari-Lari, D. M. Muzny, J. Lu, F. Lu, C. E. Lilley, S. Spanos, T. Malley and R. A. Gibbs. A gene-rich cluster between the CD4 and triosephosphate isomerase genes at human chromosome 12p13. Genome Research 6: 314-326, 1996). The C825T polymorphism corresponds for example to the genomic sequence C5500T.

The base exchange A3882C:the sequence within intron 9 ttcectagec ctttcttact gtattttttt tttttttttt tttttttttg agacagagtc is therefore mutated into the following base sequence:

ttccctagcc ctttcttact gtcttttttt tttttttttt tttttttttg agacagagtc

The base exchange G5177A:the sequence within intron 9 gctgtatagt gcagagcggg cgaggggcat agggaagtca is therefore mutated into the following base sequence:

gctgtatagt gcagagcagg cgaggggcat agggaagtca

The base exchange G5249A: the sequence within intron 9 ttctcacccc aaaccaaggg agggacaggca gggaggctg agageagcgg is therefore mutated into the following base sequence:

ttctcacccc aaaccaagga agggacaggca gggaggctg agagcagcgg

The CACA insert: the sequence within intron 10 cccccacac accacatac acacacacac ccacacaccc acacatacac ttacacgcat is therefore mutated into the following base sequence

ccccccacac acccacatac acacacacac ccacacacac acccacacat acacttacac gcat (see also FIG. 1)

The following sequences are especially important (haplotypes)

Sequence with mutations 825C-1429C-3882A-5249G and without the CACA insert in intron 10

Sequence with mutations 825T-1429T-3882C-5249G and with the CACA insert in intron 10

It was furthermore found that all the studied individuals with a 825T allele and/or with a 1429T allele at the same time carry a 3882C allele, a 5249A allele and a CACA insert. Thus there is almost 100 percent coupling equilibrium between position 825 and 1429 of the cDNA and positions 3882 and 5249 of the DNA sequences within intron 9 and the CACA insert at position 6496 of the DNA sequences within intron 10. Thus, especially genotyping at positions 3882 (intron 9) and/or 5249 (intron 9) and/or 6496 (intron 10) as well as genotyping at the cDNA positions 825 and 1429 allow prediction of an alternative splice process which leads to alternative splicing of exon 9 and 10.

The subject matter of the invention is furthermore a process for determining disease dispositions, and all sequences and variants of GNB3 can be genotyped by the individual mutation up to all possible variants (including any absolute number of variants which can be incorporated) and allow the corresponding conclusions about disease dispositions.

The process is characterized in that the DNA of a proband is isolated and genotyped at least on one of the exchanged positions and compared hereafter to the reference DNA sequence. Embodiments are preferred in which at least position 3882, position 5177, position 5249 or position 6496, at least the two positions 825 and 3882, 825 and 5177, 825 and 5249, or 825 and 6496, at least the three positions 825, 3882 and 5249, 825, 3882 and 6496, 825, 5249 and 6496, at least the four positions 825, 3882, 5249 and 1429, and 825, 3882, 5249 and 6496 and at least the five positions 825, 3882, 5249, 1429 and 6496 are genotyped.

Genotyping takes place by sequencing or by other methods which are suitable for detection of point mutations. They include PCR-supported genotyping processes such as for example allele-specific PCR, PCR reactions and restriction fragment length analysis, PCR reactions using the Taqman system or molecular beacons, genotyping processes using oligonucleotides (examples here would be dot-blotting and hybridizing, single nucleotide primary extension analysis, oligonucleotide ligation assays), processes using restriction enzymes and single nucleotide polymorphism (SNP) analysis by means of matrix-assisted laser desorption/ionization mass spectrometry (MALDI) and fundamentally any method available in the future for variant detection including gene chip technology in all its technological versions.

On this basis the process as claimed in the invention is suited for determining a wide spectrum of the most varied disease dispositions ex vivo.

In one version, determination is intended for clarifying a disposition to high blood pressure, prediction of individual blood pressure values as such, and other cardiovascular diseases, including myocardial infarction, cardiac insufficiency and stroke as well as cardiac arrhythmia. In a wider sense the formation of terminal renal insufficiency (need for dialysis) or the risk of mortality during dialysis is included.

In another embodiment the disposition to metabolic diseases such as overweight, obesity and type II diabetes mellitus, including prediction of the weight range as such or a disposition to weight changes, including prediction of the ratio of body mass as such, as are expressed in the body mass index (BMI), is ascertained.

In addition, the process is used to predict weight development after radical personal events, for example after giving birth for women or in convalescence after serious diseases. Furthermore, the process is used to estimate possible birth weight and weight development after birth. Moreover, the process is used to predict premature births, weight development of premature infants, risk of preeclampsia/gestosis, without or without the HELLP syndrome, abortions, and success of in-vitro fertilization.

In another version there is diagnostics for the disposition to altered immune reactions. This includes prediction of the reaction to inoculations, the course of infectious and autoimmune diseases, and rejection of transplanted organs. Infectious and immune diseases here are for example HIV infection and the progression to AIDS, Kaposi's sarcoma, hepatitis C and B, allergic asthma and other atopic diseases such as Crohn's disease, ulcerous colitis, atopic dermatitis and psoriasis. Furthermore, diseases of the rheumatic type, for example rheumatoid arthritis, should be mentioned here.

In another version diagnostics takes place with reference to the predisposition to the formation of malignant tumors and their course, especially a tendency to metastasis, the tendency to re-occurrence of tumors, and the tendency to tumor progression. These tumors include especially breast cancer, ovarian cancer, uterine cancer, colorectal cancer, stomach, liver, biliary and pancreatic cancer, cancer of the small intestine, malignant tumors of the skin, all forms of leukemia, all forms of lymphomas including Hodgkin's and Non-Hodgkin's lymphoma, Kaposi's sarcoma, lung cancer and cancer of the larynx, bladder, prostate and renal cell carcinoma, testicular cancer, all forms of brain tumors, for example, glioblastoma, astrocytoma, etc.

It is known that activation of heterotrimeric G proteins is decisively involved in the migration of tumor cells. This migration is responsible for metastasis. This was demonstrated among others for migration of bladder tumor cells which is greatly inhibited by the pertussis toxin (Luemmen G et al., Identification of G protein-coupled receptors potently stimulating migration of human transitional-cell carcinoma cells. Naunyn Schmiedebergs Arch Pharmacol. December 1997; 356(6): 769-76.) Here attempts are being made to therapeutically inhibit these Gi proteins by intravesicular administration of pertussis toxin (Otto T et al: Intravesical therapy with pertussis toxin before radical cystectomy in patients with bladder cancer: a Phase I study. Urology. September 1999; 54(3):458-60). There is an almost identical observation with respect to the importance of G proteins for prostate cancer (Essential role for G proteins in prostate cancer cell growth and signaling. J. Urol. December 2000; 164(6):2162-7) and for astrocytoma cells (Hemadez M. et al.; Lysophosphatidic acid inhibits Ca2+ signalling in response to epidermal growth factor receptor stimulation in human astrocytoma cells by a mechanism involving phospholipase Cγ and a Gαi protein. J Neurochem. October 2000; 75(4):1575-82). Inhibition of G protein activation also inhibits proliferation of breast cancer cells (Wickramasinghe N S, Jo H, McDonald J M, Hardy R W. Stearate inhibition of breast cancer cell proliferation. A mechanism involving epidermal growth factor receptor and G-proteins. Am J Pathol. March 1996; 148 (3):987-95).

Conversely,it is known that expression of overly activatable G proteins as is the case in artificially produced, constitutionally active α subunits produces a tumor-like cellular phenotype (N. Xu, T. Voyno Yasenetskaya, and J. S. Gutkind. Potent transforming activity of the G13 α subunit defines a novel family of oncogenes. Biochem. Biophys. Res. Commun. 201:603-609, 1994; T. A. Voyno-Yasenetskaya, A. M. Pace, and H. R. Bourne. Mutant a subunits of G12 and G13 proteins induce neoplastic transformation of Rat-1 fibroblasts. Oncogene 9:2559-2565, 1994). In addition, in a few rare cases it was found that in many tumors somatic mutations in the (x subunits of heterotrimeric G proteins can be present (J. Lyons, C. A. Landis, G. Harsh, L. Vallar, K. Gruenewald, H. Feichtinger, Q. -Y. Duh, O. H. Clark, E. Kawasaki, H. Boume, and F. McCormick. Two G protein oncogenes in human endocrine tumors. Science 249:655-659, 1990).

In summary, it can thus be stated that heterotrimeric G proteins can be assigned a decisive role in the transformation to tumor cells, but also in the migration and proliferation of tumor cells.

In any case, for a long time no generally useful genetic markers had been found which enabled predictability with respect to tumor formation and/or progression or the course of the tumor disease. Based on these observations and known findings it was as little predictable in which isoform of heterotrimeric G proteins one such generally usable genetic marker can be found.

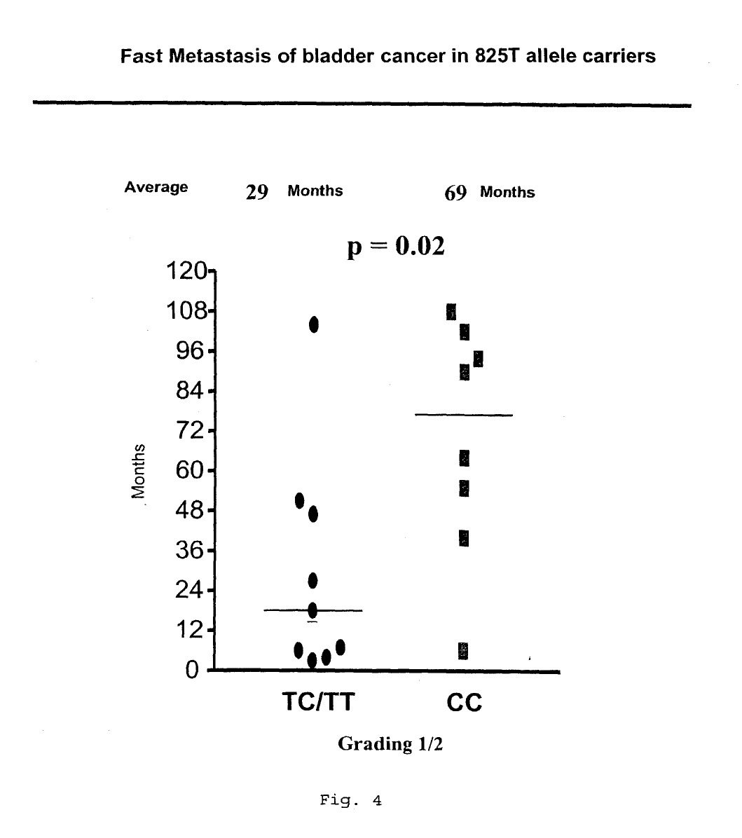

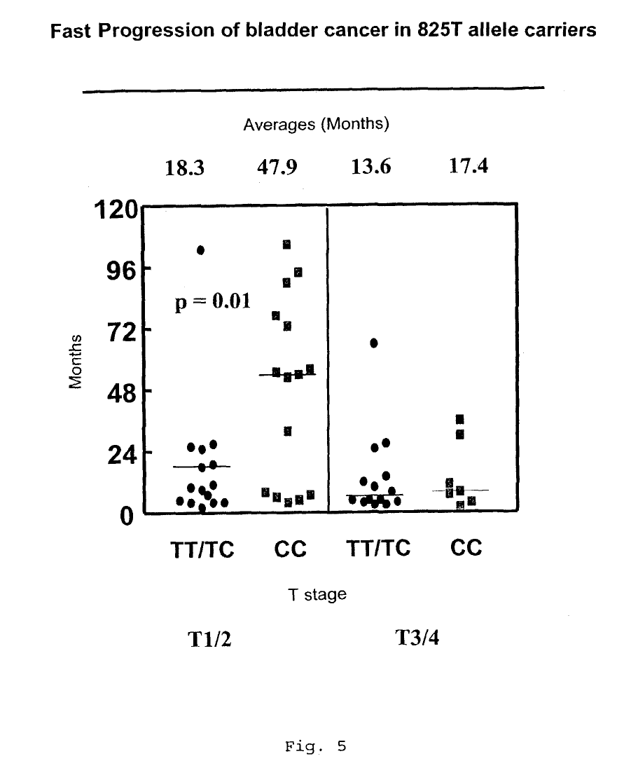

FIG. 4 demonstrates the outstanding role of mutations in gene GNB3 for the progression of human tumors using the example of bladder cancer. Here patients with bladder cancer were genotyped with respect to the C825T polymorphism, and the course of the disease was followed for 10 years. It is found that in the 825T allele carrier and in the presence of a still differentiated tumor (grading 1-2) metastasis occurs on the average within 29 months, while metastases in the homozygotic C825 allele carrier on the average occur only after 69 months. Similarly, the time from initial diagnosis of the tumor until the occurrence of progression (=recurrence or metastases) can be described in that this event occurs especially early in 825T allele carrier (FIG. 5). In less infiltrating tumors (T1/2 tumors) for 825T allele carriers the time until progression is on the average 18.3 months, in homozygotic allele C825 carriers this time on the other hand is 47.9 months. Here reference should be made especially to the fact that in almost all 825T allele carriers tumor progression occurs within two years after-initial diagnosis.

Since the cellular mechanisms for proliferation and migration (metastasis) regardless of the type of tumor or its location in the body always include activation of heterotrimeric G proteins, the phenomenon described here can be generalized. Thus, polymorphisms in gene GNB3 can be used in general as the diagnostic principle in all types of tumors.

In addition, it can be directly derived that mutations or polymorphisms in gene GNB3 can be used to predict responsiveness to tumor therapy. This applies to all chemotherapeutic agents which intervene in the widest sense in the cell cycle and inhibit the multiplication of rapidly proliferating cells in a relatively nonspecific manner. It is known that cells of individuals who carry the 825T allele are increasingly observed in the mitosis phase of the cell cycle (Rosskopf D, Schroder K J, Siffert W., Role of sodium-hydrogen exchange in the proliferation of immortalized lymphoblasts from patients with essential hypertension normotensive subjects. Cardiovasc Res. February 1995; 29(2):254-9). In addition, it can be predicted that new tumor therapeutic agents, for example antibodies, cGp nucleotides and inhibitors of signal transduction in general, for example tyrosine kinase inhibitors, in 825T allele carriers act differently than in C825 allele carriers. This can be attributed to the fact that increased activatability of G proteins, as can be observed in 825T allele carriers, also activates downstream signal transduction paths (ras- raf- MAP-kinase path): P13 kinase activation, activation of protein kinases and phospholipases, etc.) to an intensified degree.

In another embodiment, diagnostics takes place with respect to the predisposition to intelligence achievement, the capacity to learn, emotional states, the tendency to addiction and psychiatric conditions such as schizophrenia, depression, etc. It is generally known that a host of processes which take place in the human brain are controlled by hormones and so-called neuro-transmitters. For example, there are drugs which inhibit the re-absorption of serotonin and/or noradrenaline into the presynaptic nerve ending. The resulting higher concentration of transmitters in the synaptic gap then influences behavior and thinking (for example, administering Sibutramine as an appetite suppressant, Imipramine as an antidepressant). Since the G protein subunit Gβ3 occurs in a high concentration in the brain, it can be assumed that gene changes in gene GNB3 have lasting effects on behavior, thinking, intelligence, emotional states, learning capacity, processing of sensory inputs (hearing, seeing, smelling, tasting, perception of pain; cold, heat). This also includes the tendency to psychiatric conditions such as schizophrenia, depression, etc. and the tendency to addiction (nicotine, alcohol, drug addiction), the tendency to violence and much more.

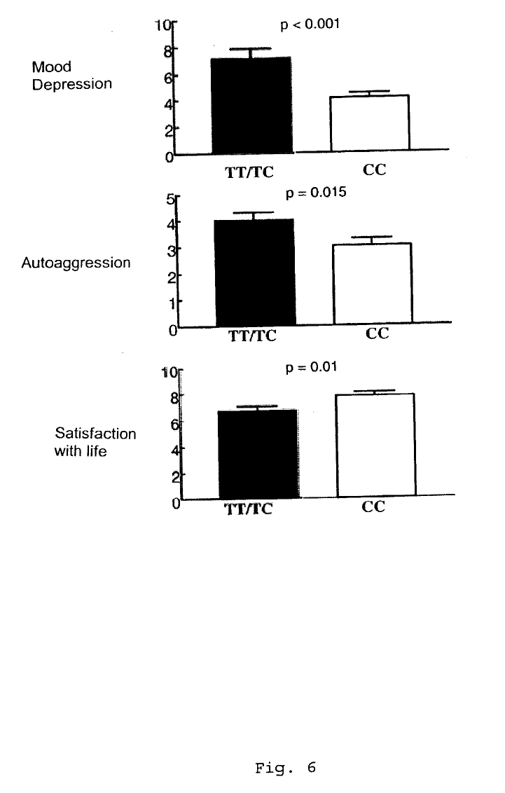

In order to fundamentally demonstrate this effect, 190 young, healthy medical students were characterized with respect to the C825T polymorphism and characterized by means of psychological test methods. For this characterization standard tests were used, such as the “Freiburger personality inventory (FPIR)”, or the “Beck depression inventory (BDI)” which are routinely used in psychiatry, psychosomatics and in psychological research to characterize personality. FIG. 6 confirms that healthy 825T allele carriers have a higher tendency to mood depression, an increased score with respect to autoaggression and a reduced score for satisfaction with life.

Thus, it is proven that fundamental processes of emotion and thinking differ in 825T allele carriers and in C825 allele carriers. Due to the signal transduction via G proteins which is intensified in a 825T allele carrier, it can moreover be deduced that genotyping in the area of the GNB3 gene can be used to predict how individuals will react to drugs and substances which influence feelings, learning capacity, psychiatric conditions, but also sense perceptions.

These predictions are also possible in healthy individuals, as demonstrated here.

Furthermore, the process also allows determination of the course and severity of diseases, and the prediction of survival times during and after serious medical conditions, for example after myocardial infarction, heart failure, stroke and/or cardiac insufficiency.

Of course the polymorphisms further described here can be used for the described diagnostics.

In particular the utility of the first teaching as claimed in the invention for pharmacogenetics will be detailed below, i.e. diagnostics of the effectiveness of drugs, potency and efficiency of drugs, and the occurrence of undesirable effects.

Principles and Objectives of Pharmacogenetics

The effectiveness of drugs and/or the occurrence of unwanted side effects is defined in addition to the specific properties of the chemically defined products by a series of parameters. Two important parameters, the attainable plasma concentration and the drug half life, to a large degree determine the effectiveness or ineffectiveness of parameters or the occurrence of unwanted effects. The plasma half-life is determined among others by the rate at which certain drugs are metabolized in the liver or other organs into effective or ineffective metabolites and at what rate they are excreted from the body, and excretion can take place via the kidneys, expired air, perspiration, spermatic fluid, stool or other bodily secretions. In addition, the effectiveness in oral administration is limited by the so-called first-pass effect, since after resorption of drugs via the intestine a certain portion is metabolized in the liver into inactive metabolites.

Mutations or polymorphisms in the genes of metabolizing enzymes can change their activity such that their amino acid composition is changed, by which the affinity for the metabolizing substrate is increased or decreased and thus the metabolism can be accelerated or slowed down. Similarly, mutations or polymorphisms in the transport proteins can change the amino acid composition such that transport and thus excretion from the body are accelerated or slowed down.

To select the substance which is optimally suited for a patient, the optimum dose, the optimum form of administration and to prevent unwanted, in part health-damaging or fatal side effects, the knowledge of genetic polymorphisms or of mutations which lead to a change of the gene products is of outstanding importance.

Action of Hormones in the Human Body and the Importance of Polymorphisms in Hormone Receptors

A host of hormones and peptide hormones of the human body exercise their effect on so-called receptors of the body cells. They are proteins of different composition. After activation of these receptors these signals must be routed into the cell interior; this is mediated via activation of heterotrimeric G proteins. These G proteins are composed of different α, β, and γ subunits. The receptors can be subdivided into certain groups depending on activatability by defined hormones. It is known to one skilled in the art that mutations or polymorphisms in certain receptors can determine the effectiveness of certain agonists or antagonist on these receptors. Thus, a frequent Gly16Arg polymorphism in the gene which codes for the β2 adrenoreceptor influences the strength of responsiveness to the β2 sympathomimetic Salbutamol (Martinez F D, et al. Association between genetic polymorphisms of the β2 adrenoreceptor and response to albuterol in children with and without a history of wheezing. J. Clin Invest. Dec. 15, 1997; 100 (12): 3184-8). Polymorphisms in the D2 receptor gene determine the frequency of the occurrence of dyskinesia in the treatment of Parkinson's disease with levadopa (Oliveri R L, et al.; Dopamine D2 receptor gene polymorphism and the risk of levadopa-induced dyskinesia in PD. Neurology. Oct. 22, 1999; 53(7): 1425-30): Polymorphisms in the μ-opiate receptor gene determine the analgesic effectiveness of opiates (Uhl G R, et. al. The μ opiate receptor as a candidate gene for pain; polymorphisms, variations in expression, nociception and opiates responses. Proc Natl Acad Sci U S A. Jul. 6, 1999; 96(14):7752-5).

The indicated gene changes in specific receptors can only be used for diagnostics of the actions of drugs such that these drugs are specific agonists or antagonists to the described receptors. Conversely, individual diagnostics of general responsiveness to drugs and individual prediction of risks of unwanted effects with drug therapy are desirable.

Diagnostics of the activatability of G proteins allows general diagnostics of the effectiveness of drugs and side effects.

Most drugs and hormones used to treat diseases, bodily malfunctions and feelings of ill-health are hormones, agonists to hormone receptors, antagonists to hormone receptors or other substances which influence the expression of receptors or the concentration of hormones directly or indirectly. A series of drugs exerts this effect in that during treatment with these substances physiological counterregulations take place which increase concentrations of hormones which activate the G protein-coupled receptors. A generally known example there is therapy with diuretics, especially loop diuretics and thiazide diuretics. The loss of salt which occurs in therapy and the reduction in blood pressure lead to activation of the renin-angiotensin-aldosterone system. The increasing formed hormone angiotensin II stimulates increased resorption of sodium in the kidneys, stimulates salt take-up, raises blood pressure by a direct vasoconstrictive effect on smooth vascular muscle cells and induces proliferation processes. It is generally known that these mechanisms caused by angiotensin II take place after coupling of the hormone to receptors which impart their action via activation of heterotrimeric G proteins. The efficiency of these effects is predictable when the strength of the activatability of G proteins can be diagnosed. Other drugs exercise their effect by inhibiting the reabsorption of the transmitters released from neurons, for example noradrenalin, adrenalin, serotonin, or dopamine. Here, one example is the drug sibutramine which inhibits the reabsorption of serotonin and noradrenalin in the central nervous system, in this way reduces the feeling of hunger and increases thermogenesis. Accordingly sibutramine can be used for treatment of adiposity. Since noradrenalin and sibutramine activate G protein-coupled receptors, the diagnostics of activatability of G proteins is especially well suited for predicting the effectiveness of sibutramine and the occurrence of typical, sibutramine-associated side effects (for example, the increase of heart rate and blood pressure).

Furthermore, the first teaching of the invention enables the prediction of which specific medications act in defined disease situations.

The object of the invention was to develop a process which in general is suitable for diagnostics of the activatability of G proteins. For this purpose, one or more polymorphisms in gene GNB3 which codes for the human Gβ3 subunit of heterotrimeric G proteins was studied. Polymorphisms are especially suited which predict the diagnosis of the occurrence or nonoccurrence of an alternative gene splicing process, by which a new splice variant of the gene and protein with at most 6 WD repeat domains is formed or its formation is suppressed. When this splice variant occurs, increased activatability of heterotrimeric G proteins and intensified activatability of all cells of the human body predictably occur. Thus, determination of the presence of polymorphisms in GNB3 allows diagnostics of the effectiveness and unwanted effects of drugs, especially agonists and antagonists of all receptors with effects which are mediated via heterotrimeric G proteins. In addition, these polymorphisms in GNB3 can be used to diagnose the effects of drugs which either directly or as a result of counterregulation mechanisms of the body raise or lower the concentrations of endogenous hormones with receptors which activate heterotrimeric G proteins. Thus, the invention allows diagnostics of effects and unwanted effects of all drugs and is not limited to drugs which influence specific receptors in an agonistic or antagonistic way.

Especially the detection of polymorphisms C825T, C1429T, A657T and G814A each alone or in any combination as haplotype analysis is used for diagnostics of increased or reduced activatability of G proteins (numbering according to cDNA, the position +1 being assigned to the start codon ATG).

In addition, all other gene changes in GNB3 which are in coupling disequilibrium to C825T or C1429T and/or promote or inhibit the alternative splicing process can be used for diagnostics. This relates especially to genetic polymorphisms in intron 9 of GNB3, for example A3882C, G5177A, G5249A and the CACA insert in intron 10 (FIG. 1).

These gene changes can be detected with any processes familiar to one skilled in the art, for example direct sequencing, restriction analysis, reverse hybridization, dot-blot or slot-27 blot processes, mass spectrometry, etc. Furthermore, these gene polymorphisms can be detected at the same time according to multiplex PCR and hybridization on a DNA chip. In addition, for diagnostics of increased activatability of G proteins other processes can also be used which use the direct detection of the formation and expression of isoforms of the Gβ3 subunit which represent splice variants.

Performing the analysis for pharmacogenetic studies includes the prediction of response to a therapy, for example with antihypertensive agents (among others, ACE inhibitors, AT1 receptor blockers, β blockers, α-receptor blockers, Ca antagonists, vasodilators), medications for treatment of cardiac insufficiency, medications for treatment of cardiac arrhythmias, asthma, depression schizophrenias, Alzheimer's disease, the use of vaccines, treatment of erectile dysfunction.

This process is especially suited for diagnostics of the action of agonists or antagonists on receptors with effects which are mediated in the known manner by G proteins. The applicability of this process has already been proven for the α2-adrenergic receptor, the fMLP, receptor and the interleukin-8 receptor (compare PCT/EP99/06534) and published (Baumgart D, et al. G protein β3 subunit 825T allele and enhanced coronary vasoconstriction on α2 adrenoreceptor activation. Circ Res. Nov. 12, 1999; 85(10):965-9; Virchow S. et al., The G protein β3 subunit splice variant Gβ3-s causes enhanced chemotaxis of human neutrophils in response to interleukin-8. Naunyn Schmiedebergs Arch Pharmacol. July 1999; 360(1):27-32; Virchow S, et al. Enhanced fMLP-stimulated chemotaxis in human neutrophils from individuals carrying the G protein subunit β3 825 T-allele. FEBS Lett. Oct. 2, 1998;436(2):155-8) and can be applied to other receptors. The following examples are named in this regard:

Adrenergic receptors, especially α and β adrenoreceptors and their isoforms and subgroups, i.e, α1 and α2 adrenoreceptors and β1, β2, β3 and β4 adrenoreceptors

Muscarine receptors and their isoforms, for example m1, m2, m3 and m4 muscarine receptors and their subtypes. Typical antagonists to muscarine receptors are for example atropine, scopolamine, ipratropium, pirenzepin and N-butyl scopolamine. Typical agonists are carbachol, bethanechol, pilocarpine, etc.

Dopamine receptors, for example D1, D2, D3, D4 and D5 receptors and their isoforms and splice variants

Serotonin receptors, for example 5-HT1, 5-HT2, 5-HT3 and 5-HT4 receptor and their subtypes. Typical agonists are Sumatriptan and Cisaprid, antagonists are for example Ondansetron, Methysergid, Buspiron and Urapidil

Endotheline receptors

Bradykinin receptors, for example B1 and B2 receptors

Angiotensin receptors, for example AT II type1 and type2 receptors, typical antagonists on the AT II receptor are Losartan and other Sartans.

Receptors for endorphins and opiates, for example the μ-opiate receptor

chemokine receptors CCR1-12 and CXCR1-8 for, for example, interleukin-1/2/3/4/5/6/7/8/9110/11/12, RANTES, MIP-1α, MIP-1β, stromal cell-derived factor, MCP1-5, TARC, lymphotactin, fractalkins, eotaxin 1-2, NAP-2, LIX, etc.

Adenosine receptors

Receptors for thrombin

Receptors for lyso-phosphatidic acid, phosphatidic acid, receptors for sphingosine phosphate and their derivatives

Receptors for prostaglandins and thromboxans, for example for PGE1, PGE2, PGF, PGD2, PGI2, PGF2α, thromboxan A2, etc.

Receptors for neuropeptides, for example NPY1-5

Histamine receptors, for example H1-H3 receptors

Receptors for platelet-activating factor (PAF receptor)

Receptors for leukotrienes

Receptors for insulin, glucagon, insulin-like growth factor (IGF-1 and IGF-2), epidermal growth factor (EGF) and platelet-derived growth factor (PDGF)

Receptors for growth hormone (GH), somatostatin (SSTR1-5, thyreotropic hormone (TSH), oxytocin, prolactin, gonadotropins

Receptors for interferons

Receptors for purines

Orphan receptors with effects mediated by G proteins.

Furthermore, the effects of drugs which influence the reabsorption, decomposition or resynthesis of neurotransmitters or in which, during therapy, changes in the expression or responsiveness of the aforementioned receptors occur (for example, Sibutramine, Fluoxetin) can be diagnosed. Moreover, the effects of all drugs which directly or indirectly change the concentrations of agonists which activate the aforementioned receptors following a physiological counterreaction can be diagnosed.

In particular, the effects and unwanted effects of the following drugs from the following indication areas can be diagnosed:

Antihypertensives, for example, β blockers (propanolol, bisoprolol, etc.), diuretics (hydrochlorothiazide and other thiazide diuretics; furosemide, piretanide and other loop diuretics, chlorthalidone, spironolactone), α1-adrenoceptor blockers (for example, Doxazosin, Prazosin), angiotensin receptor blockers (for example, Losartan), ACE inhibitors, (Enalapril, Captopril, Ramipril, etc.), Ca²⁺ channel blockers (for example, Nifedipin, Verapamil, Amlodipin, Felodipin), Clonidin, reserpine

Drugs for treatment of cardiac insufficiency, for example, β blockers (for example propanolol, metoprolol), ACE inhibitors, (Captopril, Enalapril, Ramipril, etc.), angiotensin receptor blockers (for example, Losartan)

Drugs for treatment of low blood pressure or cardiac insufficiency, for example, α and β sympathomimetics (Etilefrin, adrenalin, noradrenalin, Dobutamine)

Drugs for treatment of migraines, for example Sumatriptan, Rizatriptan, Zolmitriptan and other agonists to serotonin receptors, β blockers (for example propanolol, timolol), ergotamine and dihydroergotamine

Analgesics of the morphine type (morphine, codeine, etc.)

Drugs for treatment of coronary cardiac disease such as adenosine, β blockers (for example propanolol, acebutolol), nitrate esters and other NO donors (for example, Molsidomin), thrombocyte aggregation inhibitors

Drugs for treatment of psychiatric conditions such as alcoholism, schizophrenia, manic-depressive diseases, psychoses, depressions (for example, Fluoxetin, Paoxetin, Imipramine, Desipramine, Doxepin, Mianserin, Trazodon, Lofepramin), anxiety syndromes (Diazepam, etc.), which influence the dopaminergic, serotonergic or adrenergic system

Drugs for treatment of Alzheimer's disease (for example, Tacrin) and for treatment of Parkinson's disease (for example, Bromocriptin, L-DOPA, Carbidopa, Biperidene, Selegilin, etc.) which influence transmitter concentrations on for example muscarinergic or dopaminergic substances

Drugs for treatment of bronchial asthma which act for example either directly as bronchodilators or as antiinflammatory, for example, Salbutamol, Terbutalin, Albuterol, theophylline, Montelukast, Zafirlukast, cromoglicinic acid, Ipratropium bromide

Drugs for treatment of motility problems of the stomach or intestine (for example, N-butylscopolamine, Pirenzepin, Metoclopramid)

Drugs for treatment of adiposity which activate either directly lipolytically active receptors, for example β3-adrenergic agonists, or are centrally active, for example, Sibutramin, or similar substances

Drugs for treatment of chronic inflammation processes or disorders of the immune system, for example, interferons, in the treatment of viral hepatitides or interleukin-2 in HIV infection

Drugs for treatment of gestosis and preeclampsia/eclampsia and HELLP syndrome

Drugs for treatment of fertility problems or for elimination of cycle disorders in women

Drugs for treatment of cardiac arrhythmias

Antidiabetic drugs (Acarbose, insulin, Troglitazone, Metformin, etc.)

Hypnotics, antiemetics, and antiepileptics

Drugs for treatment of erectile dysfunction (Sildenafil, prostaglandin E1, Apomorphin).

Furthermore, gene changes in gene GNB3, for example, the C825T polymorphism, can be used to predict the responsiveness of a proband to oligonucleotides with CpG motifs. The nucleotides can be administered to activate the immune system of a patient (Krieg A M. Immune effects and mechanisms of action of CpG motifs. Vaccine, Nov. 8, 2000; 19(6):618-622). Thus, tumor diseases, but also immunological diseases, for example asthma, can be treated. Since in probands with gene changes in gene GNB3 there is generally increased activatability of immune cells genetically induced (for example S. Virchow, N. Ansorge, D. Rosskopf, H. Ruebben, and W. Siffert. The G protein β3 subunit splice variant Gβ3-s causes enhanced chemotaxis of human neutrophils in response to interleukin-8. Naunyn Schmiedebergs Arch Pharmacol. July 1999; 360(1):27-32; 1999), they also respond to this immune stimulation with CpG oligonucleotides with an intensified immune reaction. In addition, it can be predicted that the generally increased cellular activatability which is present in patients with the indicated gene changes also leads to the target cells of these patients (for example, tumor cells) reacting more strongly to activated immune cells and their released products.

Moreover, by determining the presence of gene changes in gene GNB3 it is possible to predict the response of these patients to treatment with oligonucleotides which acquire a CpG motif.

According to a second and third teaching, the invention relates to use of a gene change in the human G protein subunit Gβ3 (Gene: GNB3for finding new disease genes and for discovering and developing new drug targets for individuals in whom one of the aforementioned polymorphisms is present in GNB3, for example A(−350)G, C825T, A3882C, G5177A, G5249A, C1429T or the CACA insert in intron 10 (FIG. 1) or other gene changes in GNB3 or combinations of the indicated allele (haplotypes) are characterized in that in their body cells signal transduction which is predictably increased or decreased by gene analysis takes place via heterotrimeric G proteins (S. Virchow, N. Ansorge, D. Rosskopf, H. Ruebben, and W. Siffert. The G protein β3 subunit splice variant Gβ3-s causes enhanced chemotaxis of human neutrophils in response to interleukin-8. Naunyn Schmiedebergs Arch Pharmacol. 360 (1):27-32; 1999. S. Virchow, N. Ansorge, H. Ruebben, G. Siffert and W. Siffert. Enhanced fMLP -stimulated chemotaxis in human neutrophils from individuals carrying the G protein β3 subunit 825 T-allele. FEBS Lett. 436 (2): 155-158, 1998. W. Siffert, D. Rosskopf, G. Siffert, S. Busch, A. Moritz, R. Erbel, A. M. Sharnia, E. Ritz. H. E. Wichmann. K. H. Jakobs, and B. Horsthemke. Association of a human G-protein β3 subunit variant with hypertension. Nat. Genet. 18 (1): 45-48, 1998).

The gene status can be demonstrated by means of suitable processes known to one skilled in the art. The predisposition to certain diseases in which increased or decreased G-protein activation is important, can take place via various mechanisms.

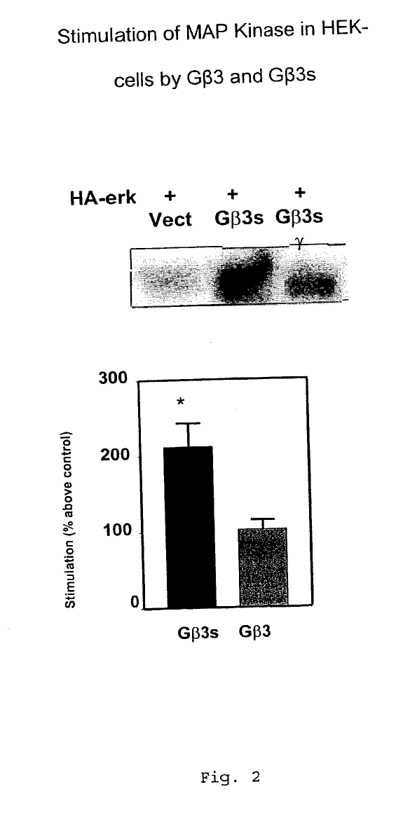

a) Individuals in whom one or more of the aforementioned gene changes occur to an increased degree form splice variants of the Gβ3 protein, for example, the previously described variants Gβ3s and Gβ3s-2 which have been described in earlier patent applications. These splice variants with at most 6 WD repeats occur as functional proteins in human cells and can be expressed in a host of expression systems by means of suitable vectors, for example in HEK-TS cells. G protein βγ subunits can biochemically interact with a host of cellular effector systems, for example with ion channels, phospholipase C—isoforms, PI3 kinase, adenylylcyclase, etc. (N. Gautam, G. B. Downes, K. Yan, and O. Kisselev. The G-protein βγ complex. Cell Signal. 10 (7): 447-455, 1998). The indicated splice variants interact with these proteins more intensively, as is shown in FIG. 2 using the activation of the MAP kinase.

First of all, HEK cells are transfected with a hemagglutinin-marked MAP kinase construct (HA-erk), the hemagglutinin being used for the specific precipitation of the construct by specific antibodies. Subsequently it is examined whether the Gβ3s splice variant compared to the wild type (Gβ3) causes intensified activation of MAP kinase. To do this the cells of the expression system are transfected in addition with vectors which code for Gβ3-s1 or Gβ3. Quantification of the activation of MAP kinase is done according to immune precipitation using standard biochemical processes known to one skilled in the art. After immune precipitation, clearly increased activation of the MAP kinase with Co transfection with Gβ3-s1 is apparent; it is almost twice as strong after Co transfection with the Gβ3 wild type. The underlying process of transfection and the detection of activation of these proteins by immune precipitation is done here with standard processes which are familiar to one skilled in the art. The result shows that these splice variants of Gβ proteins with only 6 WD repeats can activate certain interaction partners to an intensified degree. This results in the consequence that for example chemicals are identified and drugs are developed which specifically influence the interactions of Gβγ dimers which contain Gβ3s or Gβ3s-2 with these effector proteins or other biochemically defined interaction partners (enzymes, ion channels, etc.). These drugs can therefore inhibit or reduce the intensified interaction of these splice variants with effector proteins and thus can be used to treat a host of disease states. The indicated process can be expanded to other interaction partners of Gβγ subunits. Moreover, different cellular transfection systems (bacteria, yeasts or mammal cells) and a host of different vectors can be used which are familiar to one skilled in the art. In addition, to identify new interaction partners the yeast-two-hybrid system or similar systems known to one skilled in the art can be used.

In this example, the identification of suitable drug targets is done by studying the interaction of Gβ3s and Gβ3s2 with known biochemical interaction partners and/or reaction paths.

b) In addition, signal transduction via heterotrimeric G proteins causes not only direct interaction of α and βγ subunits with specific components of intracellular signal transduction which lead to a prompt influence on the effector system of a cell which does not require gene transcription, for example opening or closing of ion channels (P.Kofuji, N. Davidson, and H. A. Lester. Evidence that neuronal G-protein-gated inwardly rectifying K⁺ channels are activated by Gβγ subunit and functions as heteromultimers. Proc. Natl. Acad. Sci. USA 92:6542-6546, 1995). Rather it is also known that G-protein mediated signal transduction can activate or inhibit transcription of a host of genes. Thus, for example, expression of defined, constitutionally active G protein α subunits leads to intensified activation of prolactin promoters (J. Tian, J. Chen, and C. Bancroft. Expression of constitutively active G₈ a-subunits in GH₃ pituitary cells stimulates prolactin promoter activity. J.Biol.Chem. 269:33-36, 1994). G-protein βγ subunits in addition control translocation of other effectors into the cell nucleus which can then control transcription there (Metjian A, Roll R L, Ma A D, Abrams C S: Agonists cause nuclear translocation of phosphatidylinositol 3-kinase gamma. A Gβγ-dependent pathway that requires the p110gamma amino terminus. J. Biol Chem Sep. 24, 1999; 274 (39): 27943-7). Increased activatability of G proteins which is present in carriers of the GNB3 825T allele and other polymorphism and haplotypes of GNB3 and which can be detected by gene analysis thus leads to increased inhibition or suppression of transcription of certain genes. This makes it possible to detect differential gene activation (quantitative or qualitative) by genotyping and after identification of the genes which have been expressed differentially, to an increased degree or reduced degree and after identification of the coded gene products (for example, proteins) to identify and define new drug targets. Using several examples the process technically required for this purpose will be described.

### EXAMPLE 1

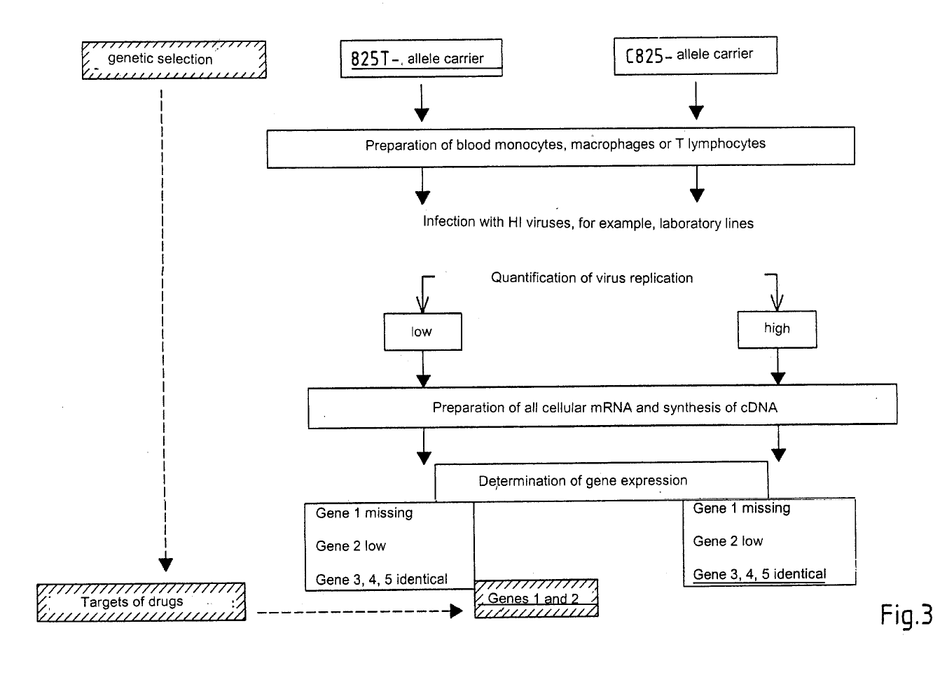

The applicant described previously (compare PCT/EP99/06534) that gene changes in gene GNB3 are associated with accelerated progression to AIDS, in these individuals likewise increased multiplication of the human immunodeficiency virus being observed. This effect is caused by increased activatability of heterotrimeric G proteins. To identify new genes or genes which have been transcribed to a reduced or increased degree the following process can for example be used; it is shown schematically in FIG. 3.

FIG. 3 shows the strategy for finding new drug targets based on genotyping on the GNB3 locus. For example, a procedure is described which can be used to develop new drugs against HIV. To do this, cells of 825T allele carriers and 825C allele carriers are inoculated with HI virus and virus multiplication is quantified. In 825T allele carriers reduced virus replication is found. From the infected cells of 825T and 825C allele carriers the total mRNA is extracted and localized in cDNA. Then a comparative quantitative and qualitative analysis of gene expression is done. Since genes 3, 4 and 5 are equally expressed, their gene products are ruled out as drug targets. Conversely the differently expressed genes 1 and 2 are potential drug targets. Their gene products are identified and they can then be used for drug screening.

First of all, to produce an expression system T lymphocytes, for example CD⁴⁺-positive T lymphocytes, or macrophages of homozygotic 825C or 825T allele carriers are isolated and cultured. Then infection with HI virus (T- or M-trope viruses) or suitable laboratory strains is done. After this infection, these viruses multiply; this can be quantified by means of processes known to one skilled in the art, for example via quantitative detection of the virus genome by means of RT-PCR, Northern Blot Analysis or detection of the p24 antigen. But here it is apparent that reduced multiplication of the HI virus in cells of homozygotic 825T allele carriers in vitro can be observed. This makes it possible to draw the conclusion that in the presence of increased signal transduction HI virus replication in vitro is reduced. This is caused by differential activation and transcription of genes in cells of 825T allele carriers in contrast to 825C allele carriers. Techniques for identification of the relevant genes which are increased, decreased or differentially activated are familiar to one skilled in the art. To do this, for example quantitative RT-PCR processes, Northern Blot analyses, hybridization on DNA chips after reverse transcription, differential display, etc. can be used. Thus genes and gene products can be identified which in the presence of a defined genotype or haplotype of gene GNB3 influence the multiplication of the HI virus. After identification of the gene products, chemicals, moreover drugs, can be developed which influence the transcription of the corresponding genes, their expression- or the function of their gene products in the desired manner. Thus the subject matter of the invention is to develop new drug targets via establishing the gene status at the GNB3 locus.

### EXAMPLE 2

It is known that 825T allele carriers have an increased risk of developing overweight and adiposity (W. Siffert et al.: Worldwide ethnic distribution of the G protein β3 subunit 825T allele and its association with obesity in Caucasian, Chinese and Black African individuals. J. Am.Soc.Nephrol. 10 (9): 1921-1930, 1999). Among those affected there is a pronounced increase of body fat (R. E. Hegele, C. Anderson, T. K. Young, and P. W. Connelly. G-protein β3 subunit gene splice variant and body fat distribution in nunavut inuit. Genome Res. 9 (10): 972-977). The fat cells of 825T allele carriers have an increased tendency to fat accumulation and reduced lipolysis, and their preadipocytes exhibit an increased tendency to profileration and to terminal differentiation. To identify the genes responsible for these processes, preadipoctyes or adipocytes after genotyping of the corresponding probands by means of standard processes for forming an expression system are placed in the cell culture. For example, terminal differentiation into adipocytes or inhibition of lipolysis can be caused at the same time by adding insulin to the culture medium. From the preadipoctyes or adipocytes of 825C or 825T allele carriers the quantitatively different or differential expression of genes for example after reverse transcription of mRNA to cDNA can be studied by means of quantitative PCR processes, Northern Blot processes, differential display, hybridization on DNA chips or similar, suitable techniques known to one skilled in the art. Identification for example of genes which have been expressed to an increased degree or differentially in 825T allele carriers leads after identification of the gene products to definition of “Drug Targets”, inhibition of which counteracts increased adipogenesis and reduced lipolysis by suitable drugs. They can be used therapeutically for drug treatment of adiposity.

Moreover the use of genotyping on the GNB3 locus as claimed in the invention and use and detection of the described variants and haplotypes represent a new process for identifying the proteins, genes and their gene products which are additionally responsible for the formation of all the aforementioned diseases and for developing suitable drugs for their treatment.

The processes described under a) and b) for determining the activatability of interaction partners or genes which are influenced in their transcription can be transferred from the gene for the β3 subunit of the human G protein to other genes.

Furthermore the invention relates to the following teachings:

A gene which contains the described intron sequence of intron 9 and intron 10 with all possible permutations for purposes of introducing the gene into human or non-human cells or tissues for causing alternative splicing and for expression of the splice variants with the objective of causing increased signal transduction.

Production of antisense constructs or medications against the described intron structure with the objective of preventing alternative splicing.

1. Use of a gene change in the gene for the β3 subunit of the human G protein, in exon 9 at position 657 in appendix 1 there being a substitution of adenine by thymine and/or in exon 10 at position 814 in appendix 1 there being a substitution of guanine by adenine and/or in the promotor area at position (−350) in appendix 1 there being a substitution of adenine by guanine and/or in intron 9 at position 3882 in appendix 1 there being a substitution of adenine by cytosine and/or in intron 9 at position 5177 in appendix 1 there being a substitution of guanine by adenine and/or in intron 9 at position 5249 in appendix 1 there being a substitution of guanine by adenine and/or in intron 10 at position 6496 in appendix 1 there being an insert consisting of the nucleotides cytosine-adenine-cytosine-adenine, for predicting physiological and pathophysiological processes in the human body

2. Use as claimed in claim 1, wherein the risk of a disease is determined.

3. Use as claimed in claim 1 or 2, wherein the course of the disease is predicted.

4. Use as claimed in one of claims 1 to 3, wherein the responsiveness of a proband to drugs is predicted.

5. Use as claimed in one of claims 1 to 4, wherein the side effects which occur in a proband when a drug is administered are predicted.

6. Use as claimed in one of claims 1 to 5, wherein in exon 10 at position 825 in appendix 1 a substitution of cytosine by thymine is present and/or wherein in exon 11 at position 1429 in appendix 1 a substitution of cytosine by thymine is present.

7. Use of at least two expression systems with at least different proportions of divergent definition of one or more genes, the activatability of an interaction partner of the proteins coded by the genes which are defined to different degrees depending on the definition of the genes being determined by the comparison of activation of the interactions partners in the expression systems.

8. Use as claimed in claim 7, wherein the identified interaction partners which can be activated depending on the definition of the genes are used a drug targets.

9. Use as claimed in claim 7 or 8, wherein the two expression systems with at least one interaction partner of the proteins coded by the differently defined genes are transfected or injected.

10. Use of at least two expression systems, especially of cells, with at least different proportions of divergent definition of one or more genes, transcription of other genes being determined depending on the definition of the differently defined genes by comparison of the transcription in the expression systems.

11. Use as claimed in claim 10, wherein the genes which have a transcription depending on the differently defined genes are used as drug targets.

12. Use as claimed in one of claims 7 to 11, wherein the gene for the β3 subunit of the G protein is used as the differently defined gene.

13. Use as claimed in one of claims 7 to 12, wherein the expression systems are human expression systems.

14. Use as claimed in one of claims 7 to 13, wherein the expression systems are stimulated chemically, for example hormonally, or physically.

15. Use as claimed in one of claims 7 to 14, wherein the expression systems are infected by pathogens, for example, viruses.

16. Use as claimed in one of claims 7 to 15, wherein the varied definition of the genes is produced by transfection or injection.
